(19) United States
(12) Patent Application Publication
(10) Pub. No.: US 2026/0144734 A1
(43) Pub. Date: May 28, 2026

(54) NATURAL HAIR STYLING COMPOSITION AND METHOD FOR STYLING HAIR

(71) Applicant: L'OREAL, Paris (FR)

(72) Inventors: Lisa Chuyin YE-TSE, Brooklyn, NY (US); Lynne MCCULLOUGH, Brooklyn, NY (US)

(73) Assignee: L'OREAL, Paris (FR)

(21) Appl. No.: 18/960,320

(22) Filed: Nov. 26, 2024

(51) Int. Cl. A61K 8/73; A61Q 5/06; A61Q 5/12

(52) U.S. Cl. CPC A61K 8/73; A61Q 5/06; A61K 2800/262; A61K 2800/81; A61K 2800/85; A61Q 5/12

(57) ABSTRACT

A hair styling composition comprising: (a) gellan gum; (b) xanthan gum; (c) starch; (d) modified cellulose; (e) polyol having from 2 to 8 carbon atoms; (f) monoalcohol having from 2 to 6 carbon atoms; and (g) water is described. The composition is a transparent, non-emulsified gel that provides hair styling and conditioning benefits to the hair. The composition is particularly useful in methods for treating hair, including methods for durably styling or shaping the hair, imparting shine, and providing quick-drying frizz control and curl definition, without flaking.

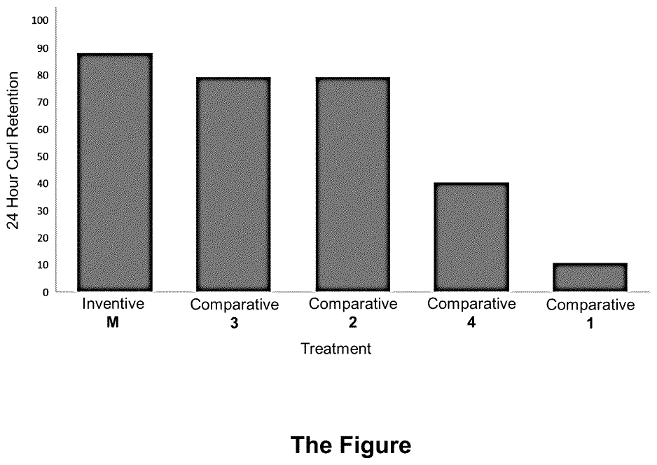

### FIELD OF THE DISCLOSURE

The present disclosure relates to hair styling compositions formulated with key natural ingredients, and to methods for treating or styling hair.

### BACKGROUND

Consumers today are increasingly seeking hair products that not only provide effective styling benefits but are also formulated with natural ingredients. There is a growing trend towards formulations that use natural, plant-derived, and sustainable ingredients, driven by concerns about environmental impact and personal health. This shift reflects a broader desire to move away from synthetic and petrochemically-derived ingredients, which are perceived as less environmentally friendly and potentially harmful.

Hair styling products vary widely in form and function, encompassing foams, gels, creams, waxes, mousses, sprays, serums, and aerosols. Each formulation caters to different styling needs and hair types, offering varying levels of benefits depending on the specific composition and application method. However, traditional styling products often suffer from drawbacks such as stickiness upon application and a stiff or “crunchy” feel after drying, which many consumers consider undesirable. For example, consumers look for styling gels that dry quickly while maintaining their desired hairstyle. However, fast-drying gels often lose transparency and shine as they dry, flaking off the hair and leaving an unattractive white residue. In contrast, slower-drying gels are better at preserving transparency and shine but tend to provide less durable hold and can feel sticky once dry.

Current advancements in hair product formulations frequently incorporate water-soluble film-forming polymers into hair styling products, many of which are synthetic polymers. These polymers exhibit diverse characteristics depending on their chemical composition; some are inherently water-soluble, while others are modified through processes like neutralization to achieve water solubility. When applied, these polymers form a film on the hair surface that can provide styling hold. However, as the solvent evaporates after application, the concentration of polymers increases, potentially leading to a film that forms unsightly residue on the hair and flaking.

The formulation of environmentally-friendly cosmetic products, which are designed and developed considering environmental issues, is becoming a major goal in an effort to meet global challenges. It is therefore essential to propose more sustainable compositions, preparation processes and ingredients, along with uses of such sustainable compositions, to address these environmental concerns. In this context, it is important to develop new cosmetic compositions with a better carbon footprint, particularly by promoting the use of renewable raw materials and/or materials with a good index of naturalness and/or materials of natural origin and, more particularly, materials of plant origin while reducing the use of compounds of petrochemical origin.

Considering the above, there remains a demand for hair styling formulations that combine the advantages of natural ingredients with exceptional styling performance, particularly those that offer quick-drying properties and durable hold without flaking.

### SUMMARY OF THE DISCLOSURE

The present disclosure describes a fast-drying hair styling composition formulated with natural key ingredients, offering a unique combination of benefits. For example, the composition dries quickly without flaking, a common problem with traditional fast-drying gels. The composition provides strong hold, maintains the desired hairstyle, and controls frizz. It also leaves the hair smooth and lightweight without reliance on synthetic film-forming polymers, cationic surfactants, and silicones. Hair styled with the composition has a clean, natural, and lightweight feel that consumers find appealing.

The hair styling composition includes:
(a) gellan gum
(b) xanthan gum;
(c) starch;
(d) modified cellulose;
(e) polyol having from 2 to 8 carbon atoms;
(f) monoalcohol having from 2 to 6 carbon atoms; and
(g) water,
   wherein all percentages by weight are based on a total weight of the hair styling composition.

The components of the hair styling composition are solubilized into a homogenous solution and therefore, the hair styling composition is not an emulsion or dispersion. The homogenous solution typically has a gel-like consistency and is translucent or transparent. Thus, the composition may be referred to as a “hair styling gel.”

Gellan gum, xanthan gum, and starch are a water-soluble polysaccharides. Gellan gum is produced by fermentation of a specific bacterium (Sphingomonas elodea). Xanthan gum is produced through the fermentation of sugars by the bacterium Xanthomonas campestris. Starch is a naturally occurring polysaccharide composed of glucose units, primarily derived from plants such as corn, potatoes, or rice. Types of starch include corn starch, rice starch, potato starch, tapioca starch, oat starch, wheat starch, arrowroot starch, and modified forms such as hydroxypropyl starch, pregelatinized starch, and acetylated distarch phosphate. Hydrolyzed starches are particularly useful and preferred. Hydrolyzed starches are created by breaking down the larger starch, thereby improving solubility. Nonlimiting examples of hydrolyzed starches include hydrolyzed corn starch, hydrolyzed rice starch, hydrolyzed potato starch, hydrolyzed tapioca starch, and hydrolyzed wheat starch.

Modified cellulose refers to cellulose that has been chemically or physically altered to enhance its solubility, functionality, or compatibility. These modifications improve properties such as viscosity, gelling, emulsification, and stabilization. Nonlimiting examples include hydroxyethyl cellulose, hydroxypropyl cellulose, methylcellulose, hydroxypropyl methylcellulose, carboxymethyl cellulose, sodium carboxymethyl cellulose, ethyl cellulose, microcrystalline cellulose, and cellulose acetate.

The hair styling composition includes one or more polyols having from 2 to 8 carbon atoms. A polyol is an alcohol characterized by having more than one hydroxyl functional group (—OH). Nonlimiting examples of polyols having from 2 to 8 carbon atoms include glycerin, ethylene glycol, propylene glycol, butylene glycol, pentylene glycol, hexylene glycol, sorbitol, mannitol, erythritol, xylitol, maltitol, isomalt, caprylyl glycol, dipropylene glycol, diglycerin, polyglycerin-3, polyglycerin-6, polyglycerin-10, neopentyl glycol, trimethylolpropane, and 1,2-octanediol. Preferred polyols having from 2 to 8 carbon atoms include glycerin, ethylene glycol, propylene glycol, butylene glycol, and dipropylene glycol.

Monoalcohols having from 2 to 6 carbon atoms are a subset of alcohols and are characterized by having only one hydroxyl functional group, distinguishing them from polyols (with multiple —OH groups). Nonlimiting examples of monoalcohols having from 2 to 6 carbon atoms include ethanol, propanol, isopropanol, butanol, isobutanol, pentanol, isopentanol, hexanol, 2-ethylhexanol, or combinations thereof. Ethanol is a particularly useful and preferred monoalcohol.

The hair styling composition has a pleasant, gel-like consistency that is easy to apply to the hair, Upon application, the composition is quick-drying and provides durable hold and styling without flaking, despite lacking synthetic thickening/film forming polymers. While synthetic thickening/film forming polymers may optionally be included, it is preferable that the hair styling composition be free or essentially free from them. This helps ensure the composition is formulated with “natural” ingredients.

A “natural” ingredient derived from sources such as plants, animals, minerals, or other naturally occurring sources—it is not petrochemically derived. These ingredients typically originate from renewable resources and can include both unprocessed materials and those that have undergone minimal modifications to enhance or modify their properties. Examples include oils, extracts, and powders from fruits, vegetables, herbs, and seeds, as well as proteins, fats, and waxes obtained from animals. Natural minerals and clays, such as kaolin and mica, are also considered natural ingredients. Modified natural products, such as hydroxyethyl cellulose and hydrolyzed starches, fall within this category, as they retain their natural origins while being processed to improve their functionality.

The viscosity of the hair styling composition will vary but is typically consistent with typical hair gel products. In various embodiments, the hair styling composition has a viscosity from about 5,000 to about 180,000 cps on a Brookfield viscometer using an RV spindle #5 at 25° C., 5 rpm.

Additional components may optionally be included in the hair styling compositions as set forth throughout the instant disclosure. These additional components, however, may also be excluded from the hair styling compositions. For example, the hair styling compositions may be free or essentially free from synthetic thickening/film forming polymers, surfactants, fatty compounds/oils, silicones, and combinations thereof.

The hair styling compositions are particularly useful in methods for styling hair, for example, human hair, especially human hair of an individual's head. For example, the compositions are useful for: (i) improving or retaining curl definition of hair; (ii) imparting humidity resistance to hair; (iii) reducing hair frizz; (iv) controlling hair volume; (v) styling or shaping hair; (vi) straightening hair; and/or (vi) improving the appearance of hair.

### DETAILED DESCRIPTION OF THE DISCLOSURE

The hair styling compositions of the instant disclosure includes a unique combination of natural ingredients, for example, gellan gum, xanthan gum, starch, and modified cellulose. The interaction or synergy amongst these components results in compositions that provide desirable softening attributes, holding ability, and styling properties to hair. More specifically, the hair styling composition includes:
(a) about 0.1 to about 3 wt. % of gellan gum
(b) about 0.1 to about 10 wt. % of xanthan gum;
(c) about 0.5 to about 20 wt. % of starch;
(d) about 0.1 to about 10 wt. % of modified cellulose;
(e) about 1 to about 30 wt. % of polyol having from 2 to 8 carbon atoms;
(f) about 10 to about 50 wt. % of monoalcohol having from 2 to 6 carbon atoms; and
(g) about 45 to about 85 wt. % of water;
   all percentages by weight based on a total weight of the composition.

The hair styling composition is preferably a gel, for example, a solubilized, homogenous gel, which means that the composition is not an emulsion or a dispersion but instead, the composition exists in a single, solubilized phase, which is substantially homogenous. The viscosity of the compositions will vary but is consistent with typical hair gel products. For example, it has a viscosity from about 5,000 to about 180,000 cps on a Brookfield viscometer using an RV spindle #5 at 25° C., 5 rpm. In addition, the composition preferably has a pH from about 6 to about 8, which is similar to the pH of healthy hair. Preferably, the hair styling compositions (the hair styling gel) is transparent or translucent.

### (a) Gellan Gum

Gellan gum is a water-soluble polysaccharide derived from the bacterium Sphingomonas elodea. Gellan gum forms gels in the presence of ions, particularly calcium, and can create a wide range of textures, from soft to firm gels. It helps create the gel-like texture of hair styling compositions while also improving long-lasting hold and style retention.

The amount of gellan gum in the hair styling compositions will vary but is typically from about 0.1 to about 3 wt. %, based on the total weight of the hair styling composition. In further embodiments, the hair styling composition includes from about 0.1 to about 2.5 wt. %, about 0.1 to about 2 wt. %, about 0.1 to about 1.5 wt. % about 0.1 to about 1 wt. %, 0.1 to about 0.8 wt. %, about 0.1 to about 0.5 wt. %, about 0.2 to about 3 wt. %, about 0.2 to about 2.5 wt. %, about 0.2 to about 2 wt. %, about 0.2 to about 1.5 wt. %, about 0.2 to about 1 wt. %, about 0.2 to about 0.8 wt. %, about 0.2 to about 0.5 wt. %, about 0.3 to about 3 wt. %, about 0.3 to about 2.5 wt. %, about 0.3 to about 2 wt. %, about 0.3 to about 1.5 wt. %, about 0.3 to about 1 wt. %, about 0.3 to about 0.8 wt. %, about 0.3 to about 0.6 wt. %, or about 0.3 to about 0.5 wt. %, based on a total weight of the hair styling composition. Preferably, the hair styling composition includes from about 0.1 to about 2.5 wt. %, more preferably about 0.1 to about 2 wt. %, and even more preferably, about 0.2 to about 1 wt. %, and even more preferably about 0.2 to about 0.8 wt. % of gellan gum, based on the total weight of the hair styling composition.

### (b) Xanthan Gum

Xanthan gum, a polysaccharide derived from sugar fermentation by Xanthomonas campestris, works synergistically with gellan gum in the hair styling compositions. When combined, the two create gels that are both firmer and well-structured while maintaining clarity, providing enhanced performance during use.

The amount of xanthan gum in the hair styling composition will vary but is typically from about 0.1 to about 10 wt. %, based on the total weight of the hair styling composition. Nonetheless, in further embodiments, the hair styling composition includes about 0.1 to about 8 wt. %, about 0.1 to about 5 wt. %, about 0.1 to about 3 wt. %, about 0.1 to about 2 wt. %, about 0.1 to about 1.5 wt. %, about 0.1 to about 1.3 wt. %, about 0.2 to about 10 wt. %, about 0.2 to about 8 wt. %, about 0.2 to about 5 wt. %, about 0.2 to about 3 wt. %, about 0.2 to about 2 wt. %, about 0.2 to about 1.5 wt. %, about 0.2 to about 1.3 wt. %, about 0.5 to about 10 wt. %, about 0.5 to about 8 wt. %, about 0.5 to about 5 wt. %, about 0.5 to about 3 wt. %, about 0.5 to about 2 wt. %, about 0.5 to about 1.5 wt. %, about 0.5 to about 1.3 wt. %, about 0.8 to about 10 wt. %, about 0.8 to about 8 wt. %, about 0.8 to about 5 wt. %, about 0.8 to about 3 wt. %, about 0.8 to about 2 wt. %, about 0.8 to about 1.5 wt. %, about 0.8 to about 1.3 wt. %, about 1 to about 10 wt. %, about 1 to about 8 wt. %, about 1 to about 5 wt. %, about 1 to about 3 wt. %, about 1 to about 2 wt. %, about 1 to about 1.5 wt. %, or about 1 to about 1.3 wt. % of xanthan gum, based on the total weight of the hair styling composition. Preferably, the hair styling composition includes from about 0.2 to about 5 wt. %, more preferably about 0.5 to about 2 wt. %, and even more preferably about 0.8 to about 1.5 wt. % of xanthan gum, based on the total weight of the hair styling composition.

In a preferred embodiment, the xanthan gum (b) is in an amount greater than or equal to the amount of the gellan gum (a).

### Combined Amount of (a) and (b)

The combined amount of the gellan gum of (a) and the xanthan gum of (b) will vary, but is typically at least about 0.5 wt. %, preferably at least about 0.8 wt. %, or more preferably, at least about 1 wt. %, based on the total weight of the hair styling composition. In further embodiments, the combined amount of (a) and (b) may be from about 1 to about 10 wt. %, about 1 to about 8 wt. %, about 1 to about 5 wt. %, about 1 to about 4 wt. %, about 1 to about 3 wt. %, about 1 to about 2 wt. %, an amount greater than 1 to about 5 wt. %, an amount greater than 1 to about 4 wt. %, an amount greater than 1 to about 3 wt. %, an amount greater than 1 to about 2 wt. %, an amount greater than 1 to about 1.8 wt. %, about 1.2 to about 5 wt. %, about 1.2 to about 4 wt. %, about 1.2 to about 3 wt. %, about 1.2 to about 3 wt. %, about 1.2 to about 2 wt. %, about 1.2 to about 1.8 wt. %, about 1.3 to about 5 wt. %, about 1.3 to about 4 wt. %, about 1.3 to about 3 wt. %, about 1.3 to about 2 wt. %, or about 1.3 to about 1.8 wt. %, based on the total weight of the hair styling composition. Preferably, the hair styling composition includes from about 1 to about 4 wt. %, more preferably about 1.2 to about 2.5 wt. %, and even more preferably about 1.3 to about 2 wt. %, based on the total weight of the hair styling composition.

### (c) Starch

Starch is a naturally occurring polysaccharide composed of glucose units, primarily derived from plants such as corn, potatoes, or rice. For purposes of the instant disclosure, the term “starch” is a generic term that includes native starch, hydrolyzed starch, and modified starch. Hydrolyzed starch refers to starch that has been broken down into smaller fragments through chemical or enzymatic processes. This increases solubility and modifies its viscosity. Modified starch, on the other hand, refers to starch that has undergone chemical, physical, or enzymatic modifications to alter its properties, such as thickening, gelling, or stability, without necessarily being hydrolyzed. This broader category includes starches modified for various applications, like crosslinked or acetylated starches.

Nonlimiting examples of native starches include corn starch, rice starch, potato starch, tapioca starch, oat starch, wheat starch, arrowroot starch. Nonlimiting examples of hydrolyzed starches include hydrolyzed corn starch, hydrolyzed rice starch, hydrolyzed potato starch, hydrolyzed tapioca starch, and hydrolyzed wheat starch. Nonlimiting examples of modified starches include acetylated starch, hydroxypropyl starch, hydroxypropyl distarch phosphate, pregelatinized starch, oxidized starch, cationic starch, crosslinked starch, maltodextrin, and dextrin. Preferably, the starch is a hydrolyzed starch, in particular, hydrolyzed corn starch.

The total amount of starch in the hair styling composition can vary but is typically from about 0.1 to about 10 wt. %, based on the total weight of the hair styling composition. In further embodiments, the hair styling composition includes about 0.01 to about 8 wt. %, about 0.1 to about 5 wt. %, about 0.1 to about 4 wt. %, about 0.1 to about 3 wt. %, about 0.2 to about 10 wt. %, about 0.2 to about 8 wt. %, about 0.2 to about 5 wt. %, about 0.2 to about 4 wt. %, about 0.2 to about 3 wt. %, about 0.5 to about 10 wt. %, about 0.5 to about 8 wt. %, about 0.5 to about 5 wt. %, about 0.5 to about 4 wt. %, about 0.5 to about 3 wt. %, about 0.8 to about 10 wt. %, about 0.8 to about 8 wt. %, about 0.8 to about 5 wt. %, about 0.8 to about 4 wt. %, about 0.8 to about 3 wt. %, about 1 to about 10 wt. %, about 1 to about 8 wt. %, about 1 to about 5 wt. %, about 1 to about 4 wt. %, about 1 to about 3 wt. %, about 2 to about 10 wt. %, about 2 to about 8 wt. %, about 2 to about 5 wt. %, about 2 to about 4 wt. %, or about 2 to about 3 wt. % of starch, based on the total weight of the hair styling composition. Preferably, the hair styling composition includes from about 0.5 to about 8 wt. %, more preferably about 1 to about 6 wt. %, and even more preferably, about 2 to about 5 wt. % of starch, preferably hydrolyzed starch.

In a preferred embodiment, the starch of (c) is in an amount greater than or equal to the combined amounts of the gellan gum of (a) and the xanthan gum of (b).

### (d) Modified Cellulose

Modified celluloses are celluloses that have been chemically or physically altered to enhance solubility, functionality, or compatibility. Common modifications include esterification, etherification, or crosslinking. These processes involve treating the cellulose with chemicals like acids, alkalis, or reagents that introduce functional groups to the cellulose molecules, altering their solubility, viscosity, and/or gelling behavior.

Nonlimiting examples of useful modified celluloses include hydroxyethyl cellulose, hydroxypropyl cellulose, methylcellulose, hydroxypropyl methylcellulose, carboxymethyl cellulose, sodium carboxymethyl cellulose, ethyl cellulose, microcrystalline cellulose, and cellulose acetate. Hydroxyethyl cellulose is a particularly useful and preferred modified cellulose.

The total amount of the modified cellulose in the hair styling compositions will vary but is typically from about 0.1 to about 10 wt. %, based on the total weight of the hair styling composition. In further embodiments, the hair styling composition includes from about 0.1 to about 8 wt. %, about 0.1 to about 5 wt. %, about 0.1 to about 3 wt. %, about 0.1 to about 2 wt. %, about 0.2 to about 10 wt. %, about 0.2 to about 5 wt. %, about 0.2 to about 3 wt. %, about 0.2 to about 2 wt. %, about 0.2 to about 1.8 wt. %, about 0.2 to about 1.5 wt. %, about 0.2 to about 1.3 wt. %, about 0.2 to about 1 wt. %, about 0.3 to about 10 wt. %, about 0.3 to about 8 wt. %, about 0.3 to about 5 wt. %, about 0.3 to about 3 wt. %, about 0.3 to about 0.3 to about 2 wt. %, about 0.3 to about 1.8 wt. %, about 0.3 to about 1.5 wt. %, about 0.3 to about 1.3 wt. %, about 0.3 to about 1 wt. %, about 0.5 to about 2 wt. %, about 0.5 to about 10 wt. %, about 0.5 to about 8 wt. %, about 0.5 to about 5 wt. %, about 0.5 to about 3 wt. %, about 0.5 to about 2 wt. %, about 0.5 to about 1.8 wt. %, about 0.5 to about 1.5 wt. %, about 0.5 to about 1.3 wt. %, about 0.5 to about 1 wt. % of modified cellulose, based on the total weight of the hair styling composition. Preferably, the hair styling composition includes from about 0.2 to about 1.8 wt. %, more preferably about 0.3 to about 1.5 wt. %, and even more preferably about 0.5 to about 1 wt. % of the modified cellulose, based on the total weight of the hair styling composition.

In a preferred embodiment, the amount of modified cellulose of (d) is less than or equal to the amount of the xanthan gum of (b).

### Combined Amount of (a), (b), and (d)

The combined amount of the gellan gum of (a), the xanthan gum of (b), and the modified cellulose of (d) will vary but is typically at least 0.5 wt. %, preferably at least 1 wt. %, and even more preferably, at least 2 wt. %, based on the total weight of the cosmetic composition. In further embodiments, the combined amount of (a), (b), and (d) is from about 0.5 to about 10 wt. %, about 0.5 to about 5 wt. %, about 0.5 to about 4 wt. %, about 0.5 to about 3 wt. %, about 0.5 to about 2.5 wt. %, about 1 to about 10 wt. %, about 1 to about 8 wt. %, about 1 to about 5 wt. %, about 1 to about 4 wt. %, about 1 to about 3 wt. %, about 1 to about 2.5 wt. %, about 1.2 to about 5 wt. %, about 1.2 to about 4 wt. %, about 1.2 to about 3 wt. %, about 1.2 to about 2.5 wt. %, about 1.5 to about 5 wt. %, about 1.5 to about 4 wt. %, about 1.5 to about 3 wt. %, about 1.5 to about 2.5 wt. %, about 2 to about 10 wt. %, about 2 to about 8 wt. %, about 2 to about 5 wt. %, about 2 to about 4 wt. %, or about 2 to about 3 wt. %, based on the total weight of the hair styling composition. Preferably, the combined amount of (a), (b), and (d) is from about 1 to about 5 wt. %, preferably about 1.5 to about 4 wt. %, even more preferably about 2 to about 3 wt. %, based on the total weight of the hair styling composition.

Ratio of (c) to (a)+(b)+(d)

The amount of the starch of (c) relative to the combined amount of the gellan gum of (a), the xanthan gum of (b), and the modified cellulose of (d) will vary. Nonetheless, the amount of the starch of (c) is typically greater than or equal to the combined amount of the gellan gum of (a), the xanthan gum of (b), and the modified cellulose of (d). In further embodiments, the weight ratio of the starch of (c) to the combined amount of the gellan gum of (a), the xanthan gum of (b), and the modified cellulose of (d) is from about 1:1 to about 4:1 ((c):((a)+(b)+(d))). Similarly, the weight ratio may be from about 1:1 to about 3:1, about 1:1 to about 2.5:1, about 1:1 to about 2:1, about 1:1 to about 1.8:1, about 1:1 to about 1.5:1 ((c):((a)+(b)+(d))).

### Additional Natural Thickening/Film-Forming Polymers

The hair styling composition do not require thickening/film-forming polymers other than the gellan gum, xanthan gum, starch, and modified cellulose. Nonetheless, one or more additional, natural thickening/film-forming polymers may optionally be included or excluded from the hair styling compositions. Nonlimiting examples of useful thickening agents/film-forming polymers include polysaccharides and proteins. Nonlimiting examples of polysaccharides include carrageenan, glucomannan, pullulan, alginate, sodium alginate, chitosan, agar, gum Arabic, hyaluronic acid, dextran, amylose, beta-glucan, konjac glucomannan, and pectin. Nonlimiting examples of proteins include silk fibroin, corn zein, collagen, albumin, keratin, wheat protein, soy protein, and rice protein.

The total amount of the additional natural thickening/film-forming polymers, if present, will vary but may be from about 0.01 to about 10 wt. %, preferably from about 0.01 to about 5 wt. %, based on the total weight of the hair styling composition. In further embodiments, the hair styling composition includes about 0.01 to about 4 wt. %, about 0.01 to about 3 wt. %, about 0.01 to about 2 wt. %, about 0.05 to about 5 wt. %, about 0.05 to about 4 wt. %, about 0.05 to about 3 wt. %, about 0.05 to about 2 wt. %, about 0.1 to about 5 wt. %, about 0.1 to about 4 wt. %, about 0.1 to about 3 wt. %, about 0.1 to about 2 wt. %, about 0.5 to about 5 wt. %, about 0.5 to about 4 wt. %, about 0.5 to about 3 wt. %, or about 0.5 to about 2 wt. % of the additional natural thickening/film-forming polymers, based on the total weight of the hair styling composition. Preferably, when the hair styling composition includes one or more additional natural thickening/film-forming polymers, the hair styling composition preferably includes about 0.01 to about 5 wt. %, more preferably about 0.05 to about 4 wt. %, and more preferably about 0.1 to about 3 wt. % of the additional natural thickening/film-forming polymers, based on the total weight of the hair styling composition.

(e) Polyols Having from 2 to 8 Carbon Atoms

A polyol is an alcohol characterized by having more than one hydroxyl functional group (—OH). Nonlimiting examples of polyols having from 2 to 8 carbon atoms include glycerin, ethylene glycol, propylene glycol, butylene glycol, pentylene glycol, hexylene glycol, sorbitol, mannitol, erythritol, xylitol, maltitol, isomalt, caprylyl glycol, dipropylene glycol, diglycerin, polyglycerin-3, polyglycerin-6, polyglycerin-10, neopentyl glycol, trimethylolpropane, 1,2-octanediol, and combinations thereof. Preferred polyols having from 2 to 8 carbon atoms include glycerin, ethylene glycol, propylene glycol, butylene glycol, and dipropylene glycol.

In various embodiments, the hair styling composition includes one or more glycols. Glycols are a group of polyols, specifically diols, characterized by having two hydroxyl (—OH) groups attached to different, adjacent carbon atoms within the same molecule. Nonlimiting examples of glycols having from 2 to 8 carbon atoms include ethylene glycol, propylene glycol, propanediol (propane-1,3-diol), butylene glycol, hexylene glycol, pentylene glycol, propanediol, and diethylene glycol. Preferred glycols include propylene glycol, propanediol, butylene glycol, diethylene glycol, and combinations thereof. Propylene glycol and butylene glycol are particularly useful glycols for the hair styling composition of the present case.

The total amount of the one or more polyols having from 2 to 8 carbon atoms in the hair styling compositions will vary but is typically from about 0.5 to about 30 wt. %, based on the total weight of the hair styling composition. In further embodiments, the hair styling composition includes from about 0.5 to about 25 wt. %, about 0.5 to about 20 wt. %, about 0.5 to about 15 wt. %, about 0.5 to about 10 wt. %, about 0.5 to about 8 wt. %, about 0.5 to about 6 wt. %, about 1 to about 30 wt. %, about 1 to about 25 wt. %, about 1 to about 20 wt. %, about 1 to about 15 wt. %, about 1 to about 10 wt. %, about 1 to about 8 wt. %, about 1 to about 6 wt. %, about 2 to about 30 wt. %, about 2 to about 25 wt. %, about 2 to about 20 wt. %, about 2 to about 15 wt. %, about 2 to about 10 wt. %, about 2 to about 6 wt. %, about 2 to about 5 wt. %, about 3 to about 30 wt. %, about 3 to about 25 wt. %, about 3 to about 20 wt. %, about 3 to about 15 wt. %, about 3 to about 10 wt. %, about 3 to about 6 wt. %, about 3 to about 6 wt. %, about 3 to about 10 wt. %, about 4 to about 8 wt. %, or about 4 to about 6 wt. % of the one or more polyols having from 2 to 8 carbon atoms, based on the total weigh to the hair styling composition. Preferably, the hair styling composition includes from about 1 to about 10 wt. %, more preferably about 2 to about 8 wt. %, and even more preferably, about 3 to about 8 wt. % of one or more polyols having from 2 to 8 carbon atoms, based on the total weight of the hair styling composition.

(f) Monoalcohols Having from 1 to 6 Carbon Atoms

Monoalcohols having from 1 to 6 carbon atoms are a subset of alcohols and are characterized by having only one hydroxyl functional group, distinguishing them from polyols (with multiple —OH groups). Nonlimiting examples of monoalcohols having from 1 to 6 carbon atoms include ethanol, propanol, butanol, pentanol, hexanol, 1-propanol (isopropanol), 1-butanol, 2-butanol, isobutanol, tert-butanol, 1-pentanol, 2-pentanol, 3-pentanol, isopentanol (isoamyl alcohol), nepentanol, 1-hexanol, 2-hexanol, 3-hexanol, cyclohexanol, and combinations thereof. In a preferred embodiment, the hair styling composition includes ethanol, isopropanol, 1-butanol, isobutanol, 1-hexanol, isoamyl alcohol, or combinations thereof. Ethanol and isopropanol are particularly preferred.

The amount of monoalcohols having from 1 to 6 carbon atoms in the hair styling compositions will vary but is typically from about 10 to about 50 wt. %, based on the total weight of the hair styling composition. In further embodiments, the hair styling composition includes from about 10 to about 40 wt. %, about 10 to about 30 wt. %, about 10 to about 25 wt. %, about 10 to about 23 wt. %, about 10 to about 20 wt. %, about 12 to about 50 wt. %, about 12 to about 40 wt. %, about 12 to about 30 wt. %, about 12 to about 25 wt. %, about 12 to about 23 wt. %, about 15 to about 50 wt. %, about 15 to about 40 wt. %, about 15 to about 30 wt. %, about 15 to about 25 wt. %, about 15 to about 23 wt. %, about 18 to about 50 wt. %, about 18 to about 40 wt. %, about 18 to about 30 wt. %, about 18 to about 25 wt. %, or about 18 to about 23 wt. %, based on the total weight of the hair styling composition. Preferably, the hair styling composition includes from about 10 to about 30 wt. %, more preferably from about 13 to about 27 wt. %, and even more preferably from about 15 to about 25 wt. % of one or more monoalcohols having from 1 to 6 carbon atoms.

### (g) Water

The total amount of water in the hair styling composition will vary but is typically from about 45 to about 85 wt. %, based on the total weight of the hair styling composition. In further embodiments, the hair styling composition includes from about 45 to about 80 wt. %, about 45 to about 75 wt. %, about 50 to about 85 wt. %, 50 to about 80 wt. %, about 50 to about 75 wt. %, about 60 to about 85 wt. %, about 60 to about 80 wt. %, about 60 to about 75 wt. %, about 65 to about 85 wt. %, about 65 to about 80 wt. %, or about 65 to about 70 wt. % of water, based on the total weight of the hair styling composition. Preferably, the hair styling includes from about 60 to about 85 wt. %, more preferably about 65 to about 80 wt. %, and even more preferably about 65 to about 75 wt. % of water, based on the total weight of the hair styling composition.

### Combined Amount of (e), (f), and (g)

The combined amount of the polyol having from 1 to 8 carbon atoms of (e), the monoalcohol having from 1 to 6 carbon atoms of (f), and the water of (g) will vary but is typically at least 50 wt. %, based on the total weight of the hair styling composition. In further embodiments, the combined amount of (e), (f), and (g) is at least about 55 wt. %, at least about 60 wt. %, at least about 65 wt. %, at least about 70 wt. %, at least about 75 wt. %, at least about 80 wt. %, at least about 85 wt. %, at least about 88 wt. %, at least about 90 wt. %, or at least about 92 wt. %, based on the total weight of the hair styling composition. The maximum value for the combined amount of (e), (f), and (g) is necessarily less than 100% due to the inclusion of the gellan gum of (a), the xanthan gum of (b), the starch of (c), and the modified cellulose of (d). Nonetheless, the maximum value for the combined amount of (e), (f), and (g) may be about 98 wt. %, about 95 wt. %, about 94 wt. %, about 93 wt. %, about 92 wt. %, about 91 wt. %, or about 90 wt. %, based on the total weight of the hair styling composition.

### (h) Alkoxylated Nonionic Surfactant

The hair styling composition optionally includes one or more nonionic surfactants, preferably, one or more alkoxylated nonionic surfactants. Alkoxylated nonionic surfactants are a class of surfactants formed by the reaction of hydrophilic molecules, such as alcohols, phenols, or fatty acids, with alkylene oxides like ethylene oxide (EO) or propylene oxide (PO). Nonlimiting examples include alkoxylated polysorbates, alkoxylated sorbitan esters, alkoxylated fatty ethers, alkoxylated fatty acids, alkoxylated glyceryl esters, or combinations thereof. In various embodiments, the alkoxylated nonionic surfactants are selected from ethoxylated fatty alcohols, polysorbates, PEG derivatives, and poloxamers.

Nonlimiting examples of ethoxylated fatty alcohols include, steareth (for example, steareth-2, steareth-20, and steareth-21), laureth (for example, laureth-4, and laureth-12), ceteth (for example, ceteth-10 and ceteth-20) and ceteareth (for example, ceteareth-2, ceteareth-10, and ceteareth-20) are mentioned. In at least one instance, the one or more alkoxylated fatty alcohols include steareth-20. In some instances, the one or more alkoxylated fatty alcohols may be exclusively steareth-20.

Polysorbates are nonionic surfactants made by the ethoxylation of sorbitan (a derivative of sorbitol) combined with fatty acids. Nonlimiting examples include polyoxyethylenated C8-C30 fatty acid esters (preferably C12-C18) of sorbitan, in particular lauric, myristic, cetylic or stearic acids, of sorbitan, especially containing from 2 to 30 mol of ethylene oxide, such as: polyoxyethylenated sorbitan monolaurate (4 EO) (Polysorbate-21) (HLB 13.3) polyoxyethylenated sorbitan monolaurate (20 EO) (Polysorbate-20) (HLB 16.7) polyoxyethylenated sorbitan monopalmitate (20 EO) (Polysorbate-40) (HLB 15.6) polyoxyethylenated sorbitan monostearate (20 EO) (Polysorbate-60) (HLB 14.9) polyoxyethylenated sorbitan monostearate (4 EO) (Polysorbate-61) (HLB 9.6) polyoxyethylenated sorbitan monooleate (20 EO) (Polysorbate-80) (HLB 15).

Nonlimiting examples of PEG derivatives include ethoxylated and hydrogenated castor oil, for example, PEG-7 hydrogenated castor oil, PEG-12 hydrogenated castor oil, PEG-20 hydrogenated castor oil, PEG-40 hydrogenated castor oil, PEG-60 hydrogenated castor oil, PEG-80 hydrogenated castor oil, and rapeseedamide ethoxylates, for example, PEG-2 rapeseedamide, PEG-4 rapeseedamide, PEG-6 rapeseedamide, PEG-10 rapeseedamide, PEG-20 rapeseedamide, PEG-30 rapeseedamide, PEG-40 rapeseedamide, PEG-60 rapeseedamide, PEG-80 rapeseedamide. Examples of ethoxylated fatty esters that may be mentioned include the adducts of ethylene oxide with esters of lauric acid, palmitic acid, stearic acid or behenic acid, and combinations thereof, especially those containing from 9 to 100 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and combinations thereof.

Poloxamers are a class of block copolymers made by combining ethylene oxide (EO) and propylene oxide (PO) units. Nonlimiting examples of poloxamers include poloxamer 188, poloxamer 407, poloxamer 338, poloxamer 237, poloxamer 908, poloxamer 124, poloxamer 181, poloxamer 184, poloxamer 331, poloxamer 105, poloxamer 103, poloxamer 188, and poloxamer 205.

In various embodiments, the one or more alkoxylated nonionic surfactants has a Hydrophobic-Lipophilic Balance (HLB) of 10 or higher. Such nonionic surfactants may be selected from alkoxylated polysorbates, alkoxylated sorbitan esters, alkoxylated fatty ethers, alkoxylated fatty acids, alkoxylated glyceryl esters, or combinations thereof. Nonlimiting examples of alkoxylated nonionic surfactants having an HLB of 10 or higher include PEG-20 stearate, PEG-40 stearate, PEG-100 stearate, PEG-20 laurate, PEG-8 laurate, PEG-40 laurate, PEG-150 distearate, PEG-7 cocoate, PEG-9 cocoate, PEG-8 oleate, PEG-10 oleate and PEG-40 hydrogenated castor oil, polysorbate 85, polysorbate 60, polysorbate 80, cetearyl glucoside, oleth-10, ceteth-10, ceteareth-20, or a mixture thereof. In a preferred embodiment, the one or more alkoxylated nonionic surfactants having an HLB of 10 or higher are selected from PEG-20 stearate, PEG-40 stearate, PEG-100 stearate, PEG-40 laurate, PEG-20 laurate, PEG-150 distearate, polysorbate 85, polysorbate 60, polysorbate 80, cetearyl glucoside, oleth-10, ceteth-10, ceteareth-20, PEG-8 laurate, PEG-9 cocoate, PEG-7 cocoate, PEG-8 oleate, PEG-40 hydrogenated castor oil, PEG-12 dimethicone, PEG-75 lanolin, PEG-150 distearate, PEG-100 lanolate, PEG-60 almond glycerides, PEG-150 PPG-2 stearate, PEG-1000 stearate, PEG-20 cetyl ether, PEG-40 castor oil, PEG-200 stearate, PEG-150 cetyl ether, PEG-4000 stearate, PEG-4 dilaurate, PEG-25 laureth-4, PEG-20 hydrogenated lanolin, and PEG-14 caprylate.

The total amount of the one or more alkoxylated nonionic surfactants, if present, will vary. Nonetheless, in various embodiments, the hair styling composition includes from about 0.01 to about 5 wt. %, based on the total weight of the hair styling composition. In further embodiments, the hair styling composition includes from about 0.01 to about 3 wt. %, about 0.01 to about 2 wt. %, about 0.01 to about 1 wt. %, about 0.05 to about 5 wt. %, about 0.05 to about 3 wt. %, about 0.05 to about 2 wt. %, about 0.05 to about 1 wt. %, about 0.1 to about 5 wt. %, about 0.1 to about 3 wt. %, about 0.1 to about 2 wt. %, about 0.1 to about 1 wt. %, about 0.5 to about 5 wt. %, about 0.5 to about 3 wt. %, about 0.5 to about 2 wt. %, or about 0.5 to about 1 wt. %, based on the total weight of the hair styling composition. Preferably, the hair styling composition includes about 0.05 to about 5 wt. %, more preferably about 0.1 to about 3 wt. %, and even more preferably about 0.5 to about 2 wt. % of one or more alkoxylated nonionic surfactants.

### (i) Alpha Hydroxy Acid Ester

The hair styling composition optionally includes one or more alpha hydroxy acid (AHA) esters. Alpha hydroxy acid esters are compounds derived from alpha hydroxy acids (AHAs), which are a class of acids characterized by a hydroxyl group (—OH) attached to the carbon adjacent to the carboxylic acid group (—COOH). In their esterified form, AHAs are reacted with alcohols to form esters, which typically have the general structure: R—O—CO—C(—OH)—R′, where R and R′ can be hydrogen or organic groups, in particular, an alkyl group having 2 to 10 carbon atoms, preferably 2 to 6 carbon atoms. Nonlimiting examples of alpha hydroxy esters include glycolic acid esters, lactic acid esters, malic acid esters, tartaric acid esters, citric acid esters, or combinations thereof.

Nonlimiting examples of glycolic acid esters include those represented by formula: CH₂OH(COOR₅), where R₅ is C₂ to C₆ alkyl group. More specific but nonlimiting examples include methyl glycolate, ethyl glycolate, n-propyl glycolate, isopropyl glycolate, butyl glycolate, or combinations thereof.

Nonlimiting examples of lactic acid esters include those represented by formula: C₂H₄OH(COOR₆), where R₆ is C₂ to C₆ alkyl group. More specific but nonlimiting examples include methyl lactate, ethyl lactate, propyl lactate, isopropyl lactate, butyl lactate, isobutyl lactate, t-butyl lactate, pentyl lactate, neopentyl lactate, isopentyl lactate, hexyl lactate, ethylhexyl lactate, glycerol lactate, benzyl lactate, or combinations thereof.

Nonlimiting examples of malic acid esters include those represented by formula: C₂H₃OH(COOR₇)(COOR₈), where R₇ and R₈ are independently H atom or C₂ to C₆ alkyl group, and one of R₇ and R₈ is an alkyl group. More specific but nonlimiting examples include dimethyl malate, diethyl malate, diisopropyl malate, dipropyl malate, dibutyl malate, diisobutyl malate, ditertbutyl malate, dimethyl acetyl malate, diethyl acetyl malate, dipropyl acetyl malate, diisopropyl acetyl malate, dibutyl acetyl malate, diisobutyl acetyl malate, ditertbutyl acetyl malate, or combinations thereof.

Nonlimiting examples of tartaric acid esters are those represented by formula: C₂H₂(OH)₂(COOR₉)(COOR₁₀), where R₉ and R₁₀ are independently H atom or C₂ to C₆ alkyl group, and one of R₉ and R₁₀ is an alkyl group. More specific but nonlimiting examples include dimethyl tartrate, diethyl tartrate, dibutyl tartrate, diisopropyl tartrate, or combinations thereof.

Nonlimiting examples of citric acid ester include those represented by formula: C₃H₄OH(COOR₁₁)(COOR₁₂)(COOR₁₃), where R₁₁, R₁₂ and R₁₃ are independently H atom or C₂ to C₆ alkyl group, and one of R₁₁, R₁₂ and R₁₃ is an alkyl group. More specific but nonlimiting examples include trimethyl citrate, triethyl citrate, tri-n-butyl citrate, tributyl citrate, acetyl triethyl citrate, acetyl tributyl citrate, trihexyl citrate, butyl trihexyl citrate, stearyl citrate, ethyl mandelate, ethyl salicylate, methyl salicylate, ethyl glycolate, or combinations thereof.

In a preferred embodiment, the one or more alpha hydroxy acid esters are selected from diethyl tartrate, ethyl lactate, triethyl citrate, or combinations thereof.

The total amount of the one or more alpha hydroxy acid esters, if present, will vary. Nonetheless, the hair styling composition may includes from about 0.1 to about 10 wt. % of the one or more alpha hydroxy acid esters, based on the total weight of the hair styling composition. In further embodiments, the hair styling composition includes from about 0.1 to about 8 wt. %, about 0.1 to about 6 wt. %, about 0.5 to about 10 wt. %, about 0.5 to about 8 wt. %, about 0.5 to about 6 wt. %, about 1 to about 10 wt. %, about 1 to about 8 wt. %, about 1 to about 6 wt. %, about 2 to about 10 wt. %, about 2 to about 8 wt. %, about 2 to about 6 wt. %, about 3 to about 10 wt. %, about 3 to about 8 wt. %, or about 3 to about 6 wt. %, based on the total weight of the hair styling composition. Preferably, the hair styling composition includes from about 0.5 to about 10 wt. %, more preferably about 1 to about 8 wt. %, and even more preferably about 2 to about 8 wt. % of one or more alpha hydroxy acid esters, based on the total weight of the hair styling composition.

### (j) Miscellaneous Ingredients

The hair styling compositions optionally include or excludes (or is essentially free from) one or more miscellaneous ingredients. In a preferred embodiment, the hair styling composition includes one or more miscellaneous ingredients. Miscellaneous ingredients are ingredients that are compatible with the hair styling compositions and do not disrupt or materially affect the basic and novel properties of the compositions. Nonlimiting examples of miscellaneous ingredients include preservatives, fragrances, pH adjusters, salts, chelating agents, amino acids (e.g., taurine), buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, particular materials, etc.) composition colorants, etc. In various embodiments, the miscellaneous ingredients are chosen from preservatives, fragrances, pH adjusters, salts, chelating agents, buffers, composition colorants, and vegetal or botanic extracts.

In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair. Further, for purposes of the instant disclosure, pearlescent agents are considered composition colorants. Pearlescent agents are cosmetic ingredients primarily used to give a shimmering effect or pearlescent effect to the composition. They can be used to enhance the visual appeal of the hair styling composition.

In addition to the above, the components described as optional components throughout the disclosure can also be considered miscellaneous ingredients if not expressly set forth as an independent component of the hair styling composition. For example, if a particular component is not expressly set forth in a claim that expressly allows for miscellaneous ingredients, such optional components should be considered included in the miscellaneous ingredients unless otherwise specified. Thus, the term “miscellaneous ingredient” is understood as a catch-all phrase representing additional components that may optionally be present in amounts designated for the miscellaneous ingredients.

The amount of the one or more miscellaneous ingredients in the hair styling composition, if present, will vary. Nonetheless, in various embodiments, the hair styling composition includes about 0.01 to about 10 wt. % of the one or more miscellaneous ingredients, based on a total weight of the hair styling composition. In further embodiments, the hair styling composition includes about 0.01 to about 8 wt. %, about 0.01 to about 6 wt. %, about 0.01 to about 5 wt. %, about 0.01 to about 3 wt. %, about 0.05 to about 10 wt. %, about 0.05 to about 8 wt. %, about 0.05 to about 6 wt. %, about 0.05 to about 5 wt. %, about 0.1 to about 10 wt. %, about 0.1 to about 8 wt. %, about 0.1 to about 6 wt. %, about 0.1 to about 5 wt. % of one or more miscellaneous ingredients, based on a total weight of the hair styling composition.

### Monosaccharides, Disaccharides, and Sugar Alcohols

The hair styling composition optionally includes (or excludes) one or more monosaccharides, disaccharides, and/or sugar alcohols. Nonlimiting examples of monosaccharides includes glucose, fructose, galactose, ribose, deoxyribose, xylose, arabinose, and mannose. Nonlimiting examples of disaccharides include sucrose, lactose, maltose, trehalose, and cellobiose. Nonlimiting examples of sugar alcohols include erythritol, xylitol, sorbitol, mannitol, isomalt, lactitol, maltitol, hydrogenated starch hydrolysates, polyglycitol syrup, arabitol, ribitol, iditol, galactitol, talitol, allitol, dulcitol, glycerol, threitol, isosorbide, sorbitan, xylitan, mannitan, maltitol syrup, glycitol, and sorbitol syrup.

The total amount of the monosaccharides, disaccharides, and/or sugar alcohols, if present, can vary. Nonetheless, if present, the monosaccharides, disaccharides, and/or sugar alcohols may be in an amount from about 0.01 to about 10 wt. %, based on the total weight of the hair styling composition. In further embodiments, the hair styling composition includes form about 0.1 to about 5 wt. %, about 0.01 to about 3 wt. %, about 0.5 to about 10 wt. %, about 0.5 to about 5 wt. %, about 0.5 to about 3 wt. %, about 1 to about 10 wt. %, about 1 to about 5 wt. %, about 1 to about 3 wt. %, about 2 to about 10 wt. %, about 2 to about 5 wt. %, about 3 to about 10 wt. %, about 3 to about 8 wt. %, or about 5 to about 10 wt. % of monosaccharides, disaccharides, and/or sugar alcohols.

In a preferred embodiment, the hair styling compositions are free or essentially free from monosaccharides.

In a preferred embodiment, the hair styling compositions are free or essentially free from disaccharides.

In a preferred embodiment, the hair styling compositions are free or essentially free from sugar alcohols.

In a preferred embodiment, the hair styling compositions are free or essentially free from monosaccharides, disaccharides, and sugar alcohols.

pH

The pH of the hair styling composition will vary but is preferably from about 5 to about 8. Nonetheless, in various embodiments, the hair styling composition has a pH of about 5 to about 7.5, about 5 to about 7, about 5 to about 6.5, about 5 to about 6, about 6 to about 8, about 6 to about 7.5, about 6 to about 7, about 6 to about 6.5, about 6.5 to about 8, about 6.5 to about 7.5, or about 6.5 to about 7. Preferably, the hair styling composition has a pH from about 5 to about 8, more preferably about 5.5 to about 7.5, and even more preferably from about 6 to about 7.

### Viscosity

The viscosity of the hair styling composition can very. Nonetheless, in various embodiments, the viscosity of the hair styling composition is similar to the viscosity of typical hair styling gels known in the art. Preferably, the hair styling gel has a viscosity of about 5,000 to about 180,000 cps on a Brookfield viscometer using an RV spindle #5 at 25° C., 5 rpm. In further embodiments, the viscosity of the hair styling composition is from about 5,000 to about 150,000 cps, about 5,000 to about 120,000 cps, about 5,000 to about 100,000 cps, about 5,000 to about 70,000 cps, about 5,000 to about 50,000 cps, about 10,000 to about 180,000 cps, about 10,000 to about 150,000 cps, about 10,000 to about 120,000 cps, about 10,000 to about 100,000 cps, about 10,000 to about 70,000 cps, about 10,000 to about 50,000 cps, 15,000 to about 180,000 cps, about 15,000 to about 150,000 cps, about 15,000 to about 120,000 cps, about 15,000 to about 100,000 cps, about 15,000 to about 70,000 cps, about 15,000 to about 50,000 cps on a Brookfield viscometer using an RV spindle #5 at 25° C., 5 rpm.

### Optional Exclusions

In various embodiments, the hair styling composition is free or essentially free from synthetic film forming polymers. Nonlimiting examples of synthetic film forming polymers include acrylic acid/acrylate-based polymers, polyvinylpyrrolidone (PVP), polyvinyl alcohol (PVA), acrylates copolymers, methacrylates copolymers, polyquaternium variants (e.g., polyquaterniums such as polyquatemium-4, -7, -11, -16, -55, -69), PVPNA copolymer, polyurethane-14, VP/DMAPA acrylates copolymer, aminomethyl propanol neutralized acrylates, carbomer, methyl methacrylate crosspolymer, sodium polystyrene sulfonate, ethyl ester of PVM/MA copolymer, adipic acid/diethylene glycol/glycerin crosspolymer, acrylates/t-butylacrylamide copolymer, and lauryl methacrylate/glycol dimethacrylate crosspolymer.

In various embodiments, the hair styling composition is free or essentially free from synthetic thickening agents, including synthetic thickening polymers. Nonlimiting examples include acrylic acid/acrylate-based polymers, carbomer, acrylates copolymers, polyacrylamide, acrylates/C10-30 alkyl acrylate crosspolymer, guar hydroxypropyltrimonium chloride, polyethylene glycol (PEG) derivatives, polyurethane thickeners, sodium polyacrylate, polyquaterniums such as polyquaternium-37, and polyvinylpyrrolidone (PVP).

In various embodiments, the hair styling composition is free or essentially free from surfactants, including anionic surfactants, cationic surfactants, amphoteric surfactants, nonionic surfactants, or combinations thereof. As noted above, the hair styling composition may optionally include one or more alkoxylated nonionic surfactants. Accordingly, in various embodiments, the hair styling composition may be free or essentially free from surfactants other than alkoxylated nonionic surfactant. Similarly, the hair styling composition may be free or essentially free from nonionic surfactants other than the one or more alkoxylated nonionic surfactants. In further embodiments, the hair styling composition may be free or essentially free from anionic surfactants. In further embodiments, the hair styling composition is free or essentially free from cationic surfactants. In other embodiments, the hair styling composition is free or essentially free from amphoteric surfactants.

In various embodiments, the hair styling composition is free from cationic conditioning polymers regardless of whether the cationic conditioning polymers are synthetic or naturally derived (e.g., guar hydroxypropyltrimonium chloride).

In various embodiments, the hair styling compositions are free from silicones, including amino-functionalized silicones.

In various embodiments, the hair styling compositions are free or essentially free from fatty compounds. Fatty compounds are a diverse group of organic molecules characterized by their insolubility in water (hydrophobic nature) and solubility in nonpolar solvents. Nonlimiting examples of fatty compounds include fatty acids, fatty alcohols, triglycerides, phospholipids, steroids, waxes, oils, glycerides, sphinoglipids, eisosanoids, alkylglycerols, polyketides, fatty acid amides, and ether lipids.

In various embodiments, the hair styling composition is free or essentially free from waxes. Nonlimiting examples of waxes that may be excluded include beeswax, carnauba wax, candelilla wax, jojoba wax, microcrystalline wax, paraffin wax, ozokerite wax, rice bran wax, sunflower wax, soy wax, shellac wax, Montan wax, lanolin wax, castor wax, bayberry wax, polyethylene wax, polyglyceryl esters, spermaceti wax, Japan wax, orange wax, palm wax, stearyl beeswax, polyethylene glycol (PEG) wax, polyglyceryl-3 beeswax, hydrogenated castor oil, ethylene glycol distearate, olive wax, mimosa wax, Myrica fruit wax, and silica silylate.

In further embodiments, the hair styling composition is free or essentially free from oils. An oil is a fatty compounds that is liquid at room temperature (˜25° C.), typically composed mainly of triglycerides (glycerol and fatty acids) or other compounds such as esters. Oils are hydrophobic, meaning they do not mix well with water.

### Methods

Another aspect of the invention pertains to methods for styling hair using the hair styling compositions described herein. The methods generally comprise applying the hair styling compositions described to hair. The hair styling compositions may be useful in a variety of settings, and either for chemically treated or untreated hair. Use on treated hair can include chemically relaxed/straightened hair or chemically dyed or bleached or lightened/highlighted hair. The hair upon which the hair styling composition is applied may be wet or damp or may be dry. In a preferred embodiment, the hair styling composition is applied to wet or damp hair. In another preferred embodiment, the hair styling composition is applied to dry hair.

The hair styling composition is typically used as a “leave-on” styling product, i.e., the hair styling composition is applied to the hair and allowed to remain on the hair without rinsing or cleansing the hair after application of the hair styling composition. For example, the hair styling composition may be applied to wet or damp hair, and the hair is allowed to dry. The hair can dry naturally or may be dried using drying tools such as a blow dryer. Also, the hair styling composition can be applied to hair (wet, damp, or dry), and the hair can be manipulated into a desired shape or style, for example using fingers, combs, or other styling tools. The hair styling composition is not rinsed or cleansed from the hair prior to manipulating the hair into the desired shape or style. The hair styling composition is also not rinsed or cleansed from the hair after manipulating the hair into the desired shape or style but instead remains on the shaped/styled hair where it continues to hold/maintain the desired shape/style for an extended period of time (e.g., throughout the day or until the hair is again cleansed according to the individual's regular cleansing and styling routine).

In various embodiments, the hair styling composition is applied to the hair immediately or shortly after cleansing the hair with a shampoo and optionally conditioning the hair with a conditioner.

### PREFERRED EMBODIMENTS

In a preferred embodiment, the hair styling composition comprises, consists essentially of, or consists of:
(a) about 0.1 to about 10 wt. %, preferably about 0.1 to about 1 wt. %, more preferably about 0.1 to about 0.8 wt. %, and more preferably about 0.2 to about 0.5 wt. % of gellan gum;
(b) about 0.1 to about 3 wt. %, preferably about 0.1 to about 2 wt. %, more preferably about 0.2 to about 1.8, and more preferably about 0.5 to about 1.5 wt. % of xanthan gum;
   provided (a) and (b) are preferably in an amount of at least 0.5 wt. %, more preferably at least 0.8 wt. %, and even more preferably, at least 1 wt. %;
(c) about 0.1 to about 10 wt. %, preferably about 1 to about 6 wt. %, and even more preferably about 2 to about 5 wt. % of starch, preferably wherein the starch is a hydrolyzed starch selected from hydrolyzed corn starch, hydrolyzed rice starch, hydrolyzed potato starch, hydrolyzed tapioca starch, hydrolyzed wheat starch, or combinations thereof;
   provided that (c) is preferably in an amount greater than or equal to a combined amount of (a) and (b);
(d) about 0.1 to about 10 wt. %, preferably about 0.1 to about 2 wt. %, more preferably about 0.1 to about 1 wt. %, and even more preferably about 0.2 to about 1 wt. % of a modified cellulose, preferably wherein the modified cellulose is selected from hydroxyethyl cellulose, hydroxypropyl cellulose, methylcellulose, hydroxypropyl methylcellulose, carboxymethyl cellulose, sodium carboxymethyl cellulose, ethyl cellulose, microcrystalline cellulose, cellulose acetate, or combinations thereof;
   provided (c) and a combined amount of (a), (b), and (d) are preferably in a weight ratio of about 1:1 to about 4:1, preferably a weight ratio of about 1:1 to about 3:1, and more preferably, a weight ratio of about 1:1 to about 2:1 ((c):((a)+(b)+(d));
(e) about 1 to about 30 wt. %, preferably about 1 to about 15 wt. %, more preferably about 2 to about 8 wt. %, and more preferably about 3 to about 7 wt. % of polyol having from 2 to 8 carbon atoms, wherein the polyol having from 2 to 8 carbon atoms is preferably selected from glycerin, propylene glycol, butylene glycol, pentylene glycol, hexylene glycol, caprylyl glycol, dipropylene glycol, or combinations thereof;
(f) about 10 to about 50 wt. %, preferably about 10 to about 30 wt. %, more preferably about 15 to about 25 wt. %, and even more preferably about 17 to about 23 wt. % of a monoalcohol having from 1 to 6 carbon atoms, wherein preferably, the monoalcohol is selected from ethanol, propanol, isopropanol, butanol, isobutanol, pentanol, isopentanol, hexanol, 2-ethylhexanol, or combinations thereof;
(g) about 45 to about 85 wt. %, preferably about 60 to about 85 wt. %, more preferably about 65 to about 80 wt. %, and even more preferably about 65 to about 75 wt. % of water;
   provided that (e), (f), and (g) are preferably in a combined amount of at least 80 wt. %, preferably at least 85 wt. %, and more preferably, at least 88 wt. %;
(h) optionally, about 0.01 to about 4 wt. %, preferably about 0.05 to about 3 wt. %, and even more preferably, about 0.1 to about 2 wt. % of an alkoxylated nonionic surfactant, wherein preferably, the alkoxylated nonionic surfactant is selected from alkoxylated polysorbates, alkoxylated sorbitan esters, alkoxylated fatty ethers, alkoxylated fatty acids, alkoxylated glyceryl esters, or combinations thereof, and even more preferably, wherein the alkoxylated nonionic surfactant has a Hydrophobic-Lipophilic Balance (HLB) of 10 or higher;
(i) optionally, about 0.1 to about 10 wt. %, preferably about 0.5 to about 8 wt. %, and more preferably about 2 to about 6 wt. % of an alpha hydroxy acid ester, wherein the alpha hydroxy acid ester is preferably selected a glycolic acid ester, a lactic acid ester, a malic acid ester, a tartaric acid ester, a citric acid ester, or combinations thereof, wherein more preferably, the alpha hydroxy acid ester is selected from diethyl tartrate, ethyl lactate, triethyl citrate, or combinations thereof;
(j) optionally, about 0.01 to about 10 wt. %, preferably about 0.1 to about 8 wt. %, and even more preferably about 1 to about 5 wt. % of miscellaneous ingredient, wherein the miscellaneous ingredient is preferably selected from preservatives, fragrances, pH adjusters, salts, chelating agents, amino acids (e.g., taurine), buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, particular materials, etc.) composition colorants, or combinations thereof;
   wherein all percentages by weight are based on a total weight of the composition; and
   wherein the composition is preferably a non-emulsified gel, which is preferably translucent or transparent.

In a further preferred embodiment, the hair styling composition comprises, consists essentially of, or consists of:
(a) about 0.1 to about 5 wt. %, preferably about 0.1 to about 1 wt. %, more preferably about 0.1 to about 0.8 wt. %, and more preferably about 0.2 to about 0.5 wt. % of gellan gum;
(b) about 0.1 to about 3 wt. %, preferably about 0.1 to about 2 wt. %, more preferably about 0.2 to about 1.8, and more preferably about 0.5 to about 1.5 wt. % of xanthan gum;
   provided (a) and (b) are preferably in an amount of at least 0.5 wt. %, more preferably at least 0.8 wt. %, and even more preferably, at least 1 wt. %; and
   preferably, (b) is in an amount greater than (a);
(c) about 0.1 to about 10 wt. %, preferably about 0.5 to about 6 wt. %, and even more preferably about 2 to about 5 wt. % of starch, preferably wherein the starch is a hydrolyzed starch selected from hydrolyzed corn starch, hydrolyzed rice starch, hydrolyzed potato starch, hydrolyzed tapioca starch, hydrolyzed wheat starch, or combinations thereof, even more preferably, wherein the starch comprises or consists of hydrolyzed corn starch;
   provided that (c) is preferably in an amount greater than or equal to a combined amount of (a) and (b);
(d) about 0.1 to about 5 wt. %, preferably about 0.1 to about 2 wt. %, more preferably about 0.1 to about 1 wt. %, and even more preferably about 0.2 to about 1 wt. % of a modified cellulose, preferably wherein the modified cellulose is selected from hydroxyethyl cellulose, hydroxypropyl cellulose, methylcellulose, hydroxypropyl methylcellulose, carboxymethyl cellulose, sodium carboxymethyl cellulose, ethyl cellulose, microcrystalline cellulose, cellulose acetate, or combination thereof, wherein more preferably, the modified cellulose comprises or consists of hydroxyethyl cellulose;
   provided (c) and a combined amount of (a), (b), and (d) are preferably in a weight ratio of about 1:1 to about 4:1, preferably a weight ratio of about 1:1 to about 3:1, and more preferably, a weight ratio of about 1:1 to about 2:1 ((c):((a)+(b)+(d));
(e) about 1 to about 35 wt. %, preferably about 1 to about 10 wt. %, more preferably about 2 to about 8 wt. %, and even more preferably about 3 to about 7 wt. % of polyol having from 2 to 8 carbon atoms, wherein the polyol having from 2 to 8 carbon atoms is preferably selected from glycerin, propylene glycol, butylene glycol, pentylene glycol, hexylene glycol, caprylyl glycol, dipropylene glycol, or combinations thereof, wherein the polyol having from 2 to 8 carbon atoms more preferably comprises or consists of glycerin, propylene glycol, butylene glycol, or combinations thereof;
(f) about 10 to about 50 wt. %, preferably about 10 to about 30 wt. %, more preferably about 15 to about 25 wt. %, and even more preferably about 17 to about 23 wt. % of a monoalcohol having from 1 to 6 carbon atoms, wherein preferably, the monoalcohol is selected from ethanol, propanol, isopropanol, butanol, isobutanol, pentanol, isopentanol, hexanol, 2-ethylhexanol, or combinations thereof, and even more preferably the monoalcohols having 1 to 6 carbon atoms comprises or consists of ethanol, isopropanol, or a combination thereof;
(g) about 45 to about 85 wt. %, preferably about 60 to about 85 wt. %, more preferably about 65 to about 80 wt. %, and even more preferably about 65 to about 75 wt. % of water;
   provided that (e), (f), and (g) are preferably in a combined amount of at least 80 wt. %, preferably at least 85 wt. %, and more preferably, at least 88 wt. %;
(h) optionally, about 0.01 to about 4 wt. %, preferably about 0.05 to about 3 wt. %, and even more preferably, about 0.1 to about 2 wt. % of an alkoxylated nonionic surfactant, wherein preferably, the alkoxylated nonionic surfactant is selected from alkoxylated polysorbates, alkoxylated sorbitan esters, alkoxylated fatty ethers, alkoxylated fatty acids, alkoxylated glyceryl esters, or combinations thereof, and even more preferably, wherein the alkoxylated nonionic surfactant has a Hydrophobic-Lipophilic Balance (HLB) of 10 or higher, wherein the alkoxylated surfactant is preferably selected from:
   polyoxyethylenated C8-C30 fatty acid esters (preferably C12-C18) of sorbitan (polysorbates), ethoxylated fatty alcohols, poloxamers, PEG derivatives such as ethoxylated and hydrogenated castor oil, for example, PEG-7 hydrogenated castor oil, PEG-12 hydrogenated castor oil, PEG-20 hydrogenated castor oil, PEG-40 hydrogenated castor oil, PEG-60 hydrogenated castor oil, PEG-80 hydrogenated castor oil, or combinations thereof;
(i) optionally, about 0.1 to about 10 wt. %, preferably about 0.5 to about 8 wt. %, and more preferably about 2 to about 6 wt. % of an alpha hydroxy acid ester, wherein the alpha hydroxy acid ester is preferably selected a glycolic acid ester, a lactic acid ester, a malic acid ester, a tartaric acid ester, a citric acid ester, or combinations thereof, wherein more preferably, the alpha hydroxy acid ester is selected from:
   glycolic acid esters represented by formula: CH₂OH(COOR₅), where R₅ is C₂ to C₆ alkyl group, preferably methyl glycolate, ethyl glycolate, n-propyl glycolate, isopropyl glycolate, butyl glycolate, or combinations thereof;
   lactic acid esters represented by formula: C₂H₄OH(COOR₆), where R₆ is C₂ to C₆ alkyl group, preferably methyl lactate, ethyl lactate, propyl lactate, isopropyl lactate, butyl lactate, isobutyl lactate, t-butyl lactate, pentyl lactate, neopentyl lactate, isopentyl lactate, hexyl lactate, ethylhexyl lactate, glycerol lactate, benzyl lactate, or combinations thereof;
   malic acid esters represented by formula: C₂H₃OH(COOR₇)(COOR₈), where R₇ and R₈ are independently H atom or C₂ to C₆ alkyl group, and one of R₇ and R₈ is an alkyl group, preferably dimethyl malate, diethyl malate, diisopropyl malate, dipropyl malate, dibutyl malate, diisobutyl malate, ditertbutyl malate, dimethyl acetyl malate, diethyl acetyl malate, dipropyl acetyl malate, diisopropyl acetyl malate, dibutyl acetyl malate, diisobutyl acetyl malate, ditertbutyl acetyl malate, or combinations thereof;
   tartaric acid esters represented by formula: C₂H₂(OH)₂(COOR₉)(COOR₁₀), where R₉ and R₁₀ are independently H atom or C₂ to C₆ alkyl group, and one of R₉ and R₁₀ is an alkyl group, preferably dimethyl tartrate, diethyl tartrate, dibutyl tartrate, diisopropyl tartrate, or combinations thereof;
   citric acid ester represented by formula: C₃H₄OH(COOR₁₁)(COOR₁₂)(COOR₁₃), where R₁₁, R₁₂ and R₁₃ are independently H atom or C₂ to C₆ alkyl group, and one of R₁₁, R₁₂ and R₁₃ is an alkyl group, preferably trimethyl citrate, triethyl citrate, tri-n-butyl citrate, tributyl citrate, acetyl triethyl citrate, acetyl tributyl citrate, trihexyl citrate, butyl trihexyl citrate, stearyl citrate, ethyl mandelate, ethyl salicylate, methyl salicylate, ethyl glycolate, or combinations thereof;
   wherein even more preferably, the alpha hydroxy acid esters is selected from diethyl tartrate, ethyl lactate, triethyl citrate, or combinations thereof;
(j) optionally, about 0.01 to about 10 wt. %, preferably about 0.1 to about 8 wt. %, and even more preferably about 1 to about 5 wt. % of miscellaneous ingredient, wherein the miscellaneous ingredient is preferably selected from preservatives, fragrances, pH adjusters, salts, chelating agents, amino acids (e.g., taurine), buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, particular materials, etc.) composition colorants, or combinations thereof;
   wherein all percentages by weight are based on a total weight of the composition; and
   wherein the composition is preferably a non-emulsified gel, which is preferably translucent or transparent.

In a further preferred embodiment, the hair styling composition comprises, consists essentially of, or consists of:
(a) about 0.1 to about 2 wt. %, preferably about 0.1 to about 1 wt. %, more preferably about 0.1 to about 0.8 wt. %, and even more preferably about 0.2 to about 0.5 wt. % of gellan gum;
(b) about 0.1 to about 5 wt. %, preferably about 0.1 to about 2 wt. %, more preferably about 0.2 to about 1.8, and even more preferably about 0.5 to about 1.5 wt. % of xanthan gum;
   provided (a) and (b) are preferably in an amount of at least 0.5 wt. %, more preferably at least 0.8 wt. %, and even more preferably, at least 1 wt. %; and
   preferably, (b) is in an amount greater than (a);
(c) about 0.1 to about 10 wt. %, preferably about 0.5 to about 6 wt. %, and even more preferably about 2 to about 5 wt. % of hydrolyzed starch selected from hydrolyzed corn starch, hydrolyzed rice starch, hydrolyzed potato starch, hydrolyzed tapioca starch, hydrolyzed wheat starch, or combinations thereof, even more preferably, wherein the starch comprises or consists of hydrolyzed corn starch;
   provided that (c) is in an amount greater than or equal to a combined amount of (a) and (b);
(d) about 0.1 to about 10 wt. %, preferably about 0.1 to about 5 wt. %, preferably about 0.1 to about 1 wt. %, and even more preferably about 0.2 to about 1 wt. % of a modified cellulose selected from hydroxyethyl cellulose, hydroxypropyl cellulose, methylcellulose, hydroxypropyl methylcellulose, carboxymethyl cellulose, sodium carboxymethyl cellulose, ethyl cellulose, microcrystalline cellulose, cellulose acetate, or combination thereof, wherein more preferably, the modified cellulose comprises or consists of hydroxyethyl cellulose;
   provided (c) and a combined amount of (a), (b), and (d) are preferably in a weight ratio of about 1:1 to about 4:1, preferably a weight ratio of about 1:1 to about 3:1, and more preferably, a weight ratio of about 1:1 to about 2:1 ((c):((a)+(b)+(d));
(e) about 1 to about 30 wt. %, preferably about 1 to about 10 wt. %, more preferably about 2 to about 8 wt. %, and more preferably about 3 to about 7 wt. % of glycerin, propylene glycol, butylene glycol, or combinations thereof;
(f) about 10 to about 50 wt. %, preferably about 10 to about 30 wt. %, more preferably about 15 to about 25 wt. %, and even more preferably about 17 to about 23 wt. % of ethanol, isopropanol, or a combination thereof;
(g) about 45 to about 85 wt. %, preferably about 60 to about 85 wt. %, more preferably about 65 to about 80 wt. %, and even more preferably about 65 to about 75 wt. % of water;
   provided that (e), (f), and (g) are preferably in a combined amount of at least 80 wt. %, preferably at least 85 wt. %, and more preferably, at least 88 wt. %;
(h) optionally, about 0.01 to about 4 wt. %, preferably about 0.05 to about 3 wt. %, and even more preferably, about 0.1 to about 2 wt. % of an alkoxylated nonionic surfactant;
(i) optionally, about 0.1 to about 10 wt. %, preferably about 0.5 to about 8 wt. %, and more preferably about 2 to about 6 wt. % of an alpha hydroxy acid ester, wherein the alpha hydroxy acid ester is preferably selected from diethyl tartrate, ethyl lactate, triethyl citrate, or combinations thereof;
(j) optionally, about 0.01 to about 10 wt. %, preferably about 0.1 to about 8 wt. %, and even more preferably about 1 to about 5 wt. % of miscellaneous ingredient, wherein the miscellaneous ingredient is preferably selected from preservatives, fragrances, pH adjusters, salts, chelating agents, amino acids (e.g., taurine), buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, particular materials, etc.) composition colorants, or combinations thereof;
   wherein all percentages by weight are based on a total weight of the composition; and
   wherein the composition is preferably a non-emulsified gel, which is preferably translucent or transparent.

### EXAMPLES

Various changes can be made in the above-compositions and methods without departing from the scope of the invention. Accordingly, it is intended that the entire disclosure in the above description and in the examples given below, is illustrative and not limiting.

### Example 1

### Inventive Hair Styling Compositions

| | | A | B | C | D | E | F | G | H | I | J | K | L |
|---|---|---|---|---|---|---|---|---|---|---|---|---|---|
| (a) | GELLAN GUM | 0.3 | 0.3 | 0.3 | 0.3 | 0.3 | 0.3 | 0.3 | 0.3 | 0.5 | 0.5 | 0.3 | 0.3 |
| (b) | XANTHAN GUM | 1.0 | 1.0 | 1.0 | 1.3 | 1.3 | 1.3 | 1.3 | 1.0 | 1.0 | 0.5 | 0.8 | 0.8 |
| Sum of (a) + (b) | | 1.3 | 1.3 | 1.3 | 1.6 | 1.6 | 1.6 | 1.6 | 1.3 | 1.5 | 1 | 1.1 | 1.1 |
| (c) | HYDROLYZED | 2.3 | 2.3 | 2.3 | 2.3 | 2.3 | 2.3 | 2.3 | 2.3 | 2.3 | 2.3 | 2.3 | 2.3 |
| | CORN STARCH | | | | | | | | | | | | |
| (d) | HYDROXYETHYL | 0.5 | 0.8 | 0.8 | 0.8 | 0.8 | 0.8 | 0.8 | 1 | 0.5 | 0.5 | 1 | 0.8 |
| | CELLULOSE | | | | | | | | | | | | |
| Sum of (a) + (b) + (d) | | 1.8 | 2.1 | 2.1 | 2.4 | 2.4 | 2.4 | 2.4 | 2.3 | 2 | 1.5 | 2.1 | 1.9 |
| Ratio (c)/((a) + (b) + (d)) | | 1.3 | 1.1 | 1.1 | 1 | 1 | 1 | 1 | 1 | 1.2 | 1.5 | 1.1 | 1.2 |
| (e) | PROPYLENE | | | | | 5 | | 5 | | | | | |
| | GLYCOL | | | | | | | | | | | | |
| | BUTYLENE | 5 | 5 | 5 | 5 | | | | 5 | 5 | 5 | 5 | 5 |
| | GLYCOL | | | | | | | | | | | | |
| | GLYCERIN | | | | | | 5 | | | | | | |
| (f) | ALCOHOL DENAT. | 15 | 15 | 20 | 20 | 20 | 20 | 20 | 15 | 15 | 15 | 15 | 15 |
| (g) | WATER | QS 100 | | | | | | | | | | | |
| (h) | PEG-40 | 0.9 | 0.9 | 0.9 | 0.9 | 0.9 | 0.9 | 0.9 | 0.9 | 0.9 | 0.9 | 0.9 | 0.9 |
| | HYDROGENATED | | | | | | | | | | | | |
| | CASTOR OIL | | | | | | | | | | | | |
| (i) | ETHYL LACTATE | 5 | 5 | | | | | | 5 | 5 | 5 | 5 | 5 |
| (j) | CITRIC ACID | 0.1 | 0.1 | 0.1 | 0.1 | 0.1 | 0.1 | 0.1 | 0.1 | 0.1 | 0.1 | 0.1 | 0.1 |
| | FRAGRANCE | 0.5 | 0.5 | 0.5 | 0.5 | 0.5 | 0.5 | 0.5 | 0.5 | 0.5 | 0.5 | 0.5 | 0.5 |

### Example 2

### Comparative Testing

TABLE 2

| | | | Comparative | | | |
|---|---|---|---|---|---|---|
| | | M | 1 | 2 | 3 | 4 |
| (a) | GELLAN GUM | 0.3 | | 0.3 | 0.3 | 0.3 |
| (b) | XANTHAN GUM | 1.3 | 1.3 | | 1.3 | 1.3 |
| Sum of (a) + (b) | | 1.6 | 1.3 | 0.3 | 1.6 | 1.6 |
| (c) | HYDROLYZED | 2.3 | 2.3 | 2.3 | | 2.3 |
| | CORN STARCH | | | | | |
| (d) | HYDROXYETHYL | 0.8 | 0.8 | 0.8 | 0.8 | |
| | CELLULOSE | | | | | |
| Sum of (a) + (b) + (d) | | 2.4 | 2.1 | 1.1 | 2.4 | 1.6 |
| Ratio (c)/((a) + (b) + (d)) | | 1:1 | 1.1:1 | 2.1:1 | NA | 1.4:1 |
| (e) | PROPYLENE | 5 | 5 | 5 | 5 | 5 |
| | GLYCOL | | | | | |
| (f) | ALCOHOL | 20 | 20 | 20 | 20 | 20 |
| | DENAT. | | | | | |
| (g) | WATER | 69 | 69 | 69 | 69 | 69 |
| (h) | PEG-40 | 0.9 | 0.9 | 0.9 | 0.9 | 0.9 |
| | HYDROGENATED | | | | | |
| | CASTOR OIL | | | | | |
| (i) | ETHYL LACTATE | | | | | |
| (j) | FRAGRANCE | 0.5 | 0.5 | 0.5 | 0.5 | 0.5 |

The hair gels listed in Table 2 were assessed for their effectiveness in maintaining hair shape/curl definition. Commercially available hair swatches (straight hair) were obtained, and the full length of the swatches was measured after pulling them tight to eliminate curls (L=full length of hair swatches). The swatches were washed with a standard shampoo, and after rinsing, 0.45 gram of either inventive hair gel M or one of the comparative gels (1-4) was applied evenly. The gels were massaged into the hair swatches to ensure uniform coverage, and the swatches were combed to align the fibers. The hair was rolled onto commercially available magnetic (plastic) rollers and allowed to dry at ambient temperature and humidity overnight. The next morning, the curled hair was removed from the rollers. After removing the curled hair swatches from the rollers, they are hung on an acrylic board. The length of the treated swatches (without pulling to remove curls) was measured and recorded (Li=length at initial assessment). Following this, the hair swatches that are hung on the acrylic board were placed in a humidity chamber set to 80% Relative Humidity (RH) and the length of the swatches assessed over a period of 24 hours. After this period, the lengths of the swatches were measured again (without pulling the hair) and recorded (Lt=length after treatment with humidity). The percentage of curl retention was calculated using the following formula:

$$Percent ( \% ) Curl Retention = [ ( L - {L}_{t} ) / ( L - {L}_{i} ) ] \times 100$$

$$where :$$

$$L = Total length of straight hair after pulling to remove curls ( maximum length ) ,$$

$${L}_{i} = Initial length of the curled hair after the application of hair gel , and$$

$${L}_{t} = Final length of the curled hair after exposure to humidity .$$

The results are illustrated in The FIGURE. The data show that hair treated with inventive gel F, which contains a combination of gellan gum, xanthan gum, hydrolyzed corn starch, and hydroxyethyl cellulose, achieved the highest curl retention, nearly 90%. In contrast, comparative gel 1, which lacks gellan gum, exhibited the poorest curl retention. Similarly, comparative gel 4, which omits hydroxyethyl cellulose, also demonstrated very low curl retention—about half that of inventive gel F. Although comparative gels 2 and 3, which lack xanthan gum and hydrolyzed corn starch respectively, performed better than gels 1 and 4, neither reached the curl retention level of inventive gel F. This outcome was unexpected, given that xanthan gum (1.3 wt. %) and hydrolyzed corn starch (2.3 wt. %) were used in greater amounts than gellan gum (0.3 wt. %) and hydroxyethyl cellulose (0.8 wt. %). The data highlight that all four polysaccharides work together synergistically to provide an impressive level of curl retention.

The foregoing disclosure illustrates and describes embodiments of the invention. The disclosure shows and describes only the preferred embodiments, but it is understood that the invention is useable in various other combinations, modifications, and environments and is capable of changes or modifications within the scope of the inventive concepts expressed herein, commensurate with the above teachings and/or the skill or knowledge of the relevant art. The embodiments described above are further intended to explain best modes known by applicant and to enable others skilled in the art to utilize the disclosure. This description is not intended to limit the invention.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the claimed invention.

The terms “a,” “an,” and “the” are understood to encompass the plural as well as the singular.

Thus, the term “a mixture thereof” or “a combination thereof” also relates to “mixtures thereof” or “combinations thereof.” Throughout the disclosure, the term “a combination thereof” may be used following a list of elements as shown in the following example where letters A-F represent the elements: “one or more elements selected from A, B, C, D, E, F, or combination thereof.” The term, “combination thereof” does not require that the combination include all of A, B, C, D, E, and F (although all of A, B, C, D, E, and F may be included). Rather, it indicates that a combination of any two or more of A, B, C, D, E, and F can be included. In other words, it is equivalent to the phrase “one or more elements selected from A, B, C, D, E, F, and a combination of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a combination thereof,” it indicates that that one or more of A, B, C, D, and F may be included, one or more of a salt of A, a salt of B, a salt of C, a salt of D, a salt of E, and a salt of F may be include, or a mixture of any two of A, B, C, D, E, F, a salt of A, a salt of B, a salt of C, a salt of D, a salt of E, and a salt of F may be included.

All percentages, parts and ratios herein are based upon the total weight of the compositions of the present disclosure, unless otherwise indicated.

The compositions and methods of the present disclosure can comprise, consist of, or consist essentially of the essential elements and limitations of the disclosure described herein, as well as any additional or optional ingredients, components, or limitations described herein or otherwise useful.

The surfactants referred to throughout the disclosure may include those having a counter-ion such as an alkali metal, alkaline earth metal, or ammonium counterion. This list of counterions, however, is non-limiting. Regardless of whether the possibility of a salt for a particular component is mentioned, both the salt and the non-salt form of the component are intended, unless stated otherwise.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions can be modified in all instances by the term “about,” meaning within +/−10% of the indicated number. For example, an amount of “about 1 wt.” can include an amount as low as 0.90 wt. % or as high as 1.1. Similarly, an amount of “about 50” can include an amount as low as 45 wt. % and as high as 55 wt. %.

The term “rinse-off” as used herein indicates that the composition is used in a context whereby the composition is ultimately rinsed or washed from the treated surface, (e.g., skin, hair, or hard surfaces) either after or during the application of the product. These rinse-off compositions are to be distinguished from compositions referred to a “leave-on” compositions. For example, a rinse-off composition is applied to the hair, optionally allowed to remain on the hair for a short time (e.g., a few second up to about 5, 10, or 15 minutes) and subsequently rinsed from the hair before the hair styled.

The compositions described throughout this disclosure may be a “leave-on” composition. A “leave-on” (also called leave-in) composition refers to a composition that is applied to skin or hair and is not subjected to immediate rinsing and/or washing, for example for at least 4 hours or for a period of time ranging from 4 hours up to 72 hours, from 4 hours up to 48 hours, or from 8 hours up to 36 hours, or from 8 hours up to 24 hours. In other words, the product is applied to the hair and remains on the hair, as styled, i.e., it is not removed from the hair prior to styling the hair.

The term “transparent” with respect to a transparent composition indicates that the composition has transmittance of at least 80% at a wavelength of 600 nm, for example measured using a Lambda 40 UV-visible spectrometer. The compositions may have, for example, a transmittance of at least 80%, at least 90%, or at least 95% at a wavelength of 600 nm, measured, for example, using a Lambda 40 UV-visible spectrometer. The term “clear” is interchangeable with the term “transparent” for purposes of the instant disclosure. A human can typically see through a transparent composition, for example, and read the text on the other side of a clear glass or clear plastic bottle containing the composition.

The term “translucent” with respect to a translucent composition indicates that the composition has a transmittance of at least 50% at a wavelength of 600 nm, for example measured using a Lambda 40 UV-visible spectrometer. A human cannot likely see through a translucent composition, for example, and read the text on the other side of a clear glass or clear plastic bottle containing the composition. Rather, the text is usually blurred and difficult or not possible to read, yet movement and structure can normally be identified.

The term “opaque” with respect to an opaque composition indicates that the composition is not transparent or translucent, i.e., has a transmittance of less than 50% at a wavelength of 600 nm, for example measured using a Lambda 40 UV-visible spectrometer.

Some of the various categories of components identified may overlap. In such cases where overlap may exist and the composition includes both components (or the composition includes more than two components that overlap), an overlapping component or ingredient does not represent more than one element of the claims. For example, if a polyacrylate falls within the description of a thickening polymer and within the description of film forming polymer, a single polyacrylate is understood as being only the thickening polymer or only the film forming polymer. A single polyacrylate cannot simultaneously be construed as both a thickening polymer and a film forming polymer in the same composition, for example, when the claims set forth both a thickening polymer and a film forming polymer (even though the polyacrylate may function as both a thickening polymer and a film forming polymer). In other words, a single compound or ingredient cannot simultaneously serve as two different components or elements of a claim.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub-ranges between, the given ranges. Thus, a range from 1-5, includes specifically 1, 2, 3, 4 and 5, as well as sub ranges such as 2-5, 3-5, 2-3, 2-4, 1-4, etc.

For purposes of the present disclosure, “natural” ingredients are substances derived from sources such as plants, animals, and minerals. These ingredients typically originate from renewable resources and can include both unprocessed materials and those that have undergone minimal modifications to enhance or modify their properties. Examples include oils, extracts, and powders from fruits, vegetables, herbs, and seeds, as well as proteins, fats, and waxes obtained from animals. Natural minerals and clays, such as kaolin and mica, are also considered natural ingredients. Modified natural products, such as hydroxyethyl cellulose and hydrolyzed starches, fall within this category, as they retain their natural origins while being processed to improve their functionality.

Processing of natural ingredients may involve methods such as extraction, hydrolysis, and fermentation. These processes enhance usability while preserving the essential qualities of the ingredients. Natural ingredients are often biodegradable and environmentally friendly, typically recognized as safe (GRAS) and biocompatible. They are rich in bioactive compounds, antioxidants, and nutrients, frequently maintaining sensory attributes like aroma, color, and texture. These ingredients serve various functions, including emollients and moisturizers like shea butter and coconut oil, preservatives from essential oils, thickeners and stabilizers like xanthan gum and guar gum, and active ingredients such as aloe vera and tea tree oil.

For purposes of the present disclosure, the term “natural polymer” is a macromolecular compound that originates from natural sources, such as plants, animals, or microorganisms, and is composed of repeating structural units (monomers) produced biologically. This category includes polymers that may undergo minimal processing or modification, such as hydrolysis or chemical alteration, to enhance their properties or functionality. Examples include hydrolyzed starch, hydroxyethyl cellulose, and other modified natural polysaccharides and proteins.

For purposes of the present disclosure, “synthetic” ingredients are ingredients created through chemical processes and do not occur naturally in their final forms. Typically manufactured in laboratories or industrial settings, these ingredients often derive from petroleum-based raw materials or other non-renewable sources. Many synthetic polymers and compounds come from petrochemicals, and their production involves controlled chemical reactions that allow for consistency in quality and performance. Synthetic ingredients are not found in nature.

For the purposes of the present disclosure, the term “synthetic polymer” is a macromolecular compound created through chemical synthesis processes, typically involving the polymerization of monomers derived from petroleum-based sources or other non-natural substrates. The polymer is neither present in nature nor a derivative of a polymer of natural origin. Synthetic ingredients are typically produced in a laboratory or industrial setting. Unlike natural ingredients, which are derived from plants, minerals, or animals, synthetic ingredients do not occur in nature in their final form. A synthetic polymer.

The drying time of the hair gel can be measured by applying a standard amount of the gel to a defined area (such as a hair strand or a flat surface) and then recording the time it takes for the gel to lose its tackiness and become dry to the touch. About 1-2 grams of hair gel is evenly applied to a designated area of hair or a flat surface. The hair upon which the gel is applied or the flat surface is maintained in a controlled environment at 25° C. and 50% relative humidity. The applied gel is periodically checked (e.g., every minute) to determine if the gel feels dry to the touch. The gel is considered dry when it no longer feels sticky or tacky. Typical hair gel products have a drying time of about 5 to 10 minutes. The hair styling compositions of the instant case dry more quickly than typical hair gel products. For example, within about 1 to 5 minutes. Preferably, upon application to the hair, the hair styling composition dries in less than 10 minutes, more preferably, in less than 5 minutes, and even more preferably in less than 3 minutes.

All components positively set forth in the instant disclosure can be negatively excluded from the claims and from the compositions and methods described throughout the disclosure. In other words, the compositions of the instant disclosure may be free or essentially free of any one or more of the components positively set forth in the instant disclosure for possible inclusion in the compositions.

The term “substantially free” or “essentially free” as used herein means the specific material may be present in small amounts that do not materially affect the basic and novel characteristics of the claimed invention. For instance, there may be less than 1% by weight of a specific material added to a composition, based on the total weight of the compositions (provided that an amount of 1% or less by weight does not materially affect the basic and novel characteristics of the claimed invention). Similarly, when a composition is essentially free from a particular element, the composition may include 1 wt. % or less, 0.5 wt. % or less, 0.1 wt. % or less, 0.05 wt. % or less, 0.01 wt. % or less, or none of the specified material. As an, if a composition is “substantially free” or “essentially free” from ethanol, the composition may optionally include ethanol in an amount up to 1 wt. %, based on the total weight of the composition, provided an amount of up to 1 wt. % does not materially affect the basic and novel characteristics of the composition. Furthermore, the composition may include 0.5 wt. % or less, 0.1 wt. % or less, 0.05 wt. % or less, 0.01 wt. % or less, or no ethanol.

All publications and patent applications cited throughout the disclosure are incorporated herein by reference in their entirety, and for any and all purposes, as if each individual publication or patent application were specifically and individually indicated to be incorporated by reference. In the event of an inconsistency between the present disclosure and any publications or patent application incorporated herein by reference, the present disclosure controls.

1. A hair styling composition comprising:
(a) about 0.1 to about 3 wt. % of gellan gum
(b) about 0.1 to about 10 wt. % of xanthan gum;
(c) about 0.5 to about 20 wt. % of starch;
(d) about 0.1 to about 10 wt. % of modified cellulose;
(e) about 1 to about 50 wt. % of polyol having from 2 to 8 carbon atoms;
(f) about 10 to about 50 wt. % of monoalcohol having from 2 to 6 carbon atoms; and
(g) about 45 to about 85 wt. % of water,
wherein all percentages by weight are based on a total weight of the composition.

2. The composition of claim 1 wherein (c) is in an amount greater than a combined amount of (a) and (b); and/or (c) and a combined amount of (a), (b), and (d) are in a weight ratio of about 1:1 to about 3:1 ((c):((a)+(b)+(d))).

3. The composition of claim 1, wherein the composition is a non-emulsified gel.

4. The composition of claim 1, wherein the starch is hydrolyzed starch.

5. The composition of claim 1, wherein the polyol having from 2 to 8 carbon atoms is selected from glycerin, propylene glycol, butylene glycol, pentylene glycol, hexylene glycol, caprylyl glycol, dipropylene glycol, or combinations thereof.

6. The composition of claim 1, wherein the modified cellulose is selected from hydroxyethyl cellulose, hydroxypropyl cellulose, methylcellulose, carboxymethyl cellulose, ethyl cellulose, cellulose acetate, hydroxybutyl cellulose, propylene glycol cellulose, sodium carboxymethyl cellulose, or combinations thereof.

7. The composition of claim 1, wherein the monoalcohol having from 2 to 6 carbon atoms is selected from ethanol, propanol, isopropanol, butanol, isobutanol, pentanol, isopentanol, hexanol, 2-ethylhexanol, or combinations thereof.

8. The composition of claim 7, wherein the monoalcohol comprises or consists of ethanol.

9. The composition of claim 1, wherein the composition is essentially free from synthetic polymers, essentially free from oils, or essentially free from both synthetic polymers and oils.

10. The composition of claim 1, wherein the compositions further comprises an alkoxylated nonionic surfactant.

11. The composition of claim 10, wherein the alkoxylated nonionic surfactant has a Hydrophobic-Lipophilic Balance (HLB) of 10 or higher.

12. The composition of claim 11, wherein the alkoxylated nonionic surfactant having an HLB of 10 or higher is selected from alkoxylated polysorbates, alkoxylated sorbitan esters, alkoxylated fatty ethers, alkoxylated fatty acids, alkoxylated glyceryl esters, or combinations thereof.

13. The composition of claim 12, wherein the alkoxylated nonionic surfactant having an HLB of 10 or higher is selected from PEG-20 stearate, PEG-40 stearate, PEG-100 stearate, PEG-20 laurate, PEG-8 laurate, PEG-40 laurate, PEG-150 distearate, PEG-7 cocoate, PEG-9 cocoate, PEG-8 oleate, PEG-10 oleate and PEG-40 hydrogenated castor oil, polysorbate 85, polysorbate 60, polysorbate 80, cetearyl glucoside, oleth-10, ceteth-10, ceteareth-20, or combinations thereof.

14. The composition of claim 1, wherein a combination of (e), (f), and (g) is at least 85 wt. %.

15. The composition of claim 1, wherein the composition has a viscosity of about 5,000 to about 180,000 cps on a Brookfield viscometer using an RV spindle #5 at 25° C., 5 rpm.

16. The composition of claim 1, wherein the composition is transparent or translucent.

17. A hair styling composition comprising:
(a) about 0.1 to about 1 wt. % of gellan gum;
(b) about 0.1 to about 2 wt. % of xanthan gum;
provided (a) and (b) are in an amount of at least 1 wt. %;
(c) about 1 to about 10 wt. % of hydrolyzed cornstarch;
provided (c) is in an amount greater than a combined amount of (a) and (b);
(d) about 0.2 to about 5 wt. % of hydroxyethyl cellulose;
provided (c) and a combined amount of (a), (b), and (d) are in a weight ratio of about 1:1 to about 2:1 ((c):((a)+(b)+(d)));
(e) about 1 to about 10 wt. % of glycerin, propylene glycol, butylene glycol, or combinations thereof;
(f) about 10 to about 25 wt. % of ethanol, propylene glycol, or combinations thereof;
(g) about 60 to about 85 wt. % of water;
provided (e), (f), and (g) are in a combined amount of at least 80 wt. %;
(h) about 0.01 to about 4 wt. % of an alkoxylated nonionic surfactant;
(i) optionally, about 0.1 to about 5 wt. % of one or more alpha hydroxy acid esters; and
(j) optionally, about 0.01 to about 5 wt. % of miscellaneous ingredient;
wherein all percentages by weight are based on a total weight of the composition.

18. A method for styling hair comprising applying the composition of claim 1 to the hair.

19. The method of claim 18, wherein the composition is applied to wet or damp hair, and after application, without rinsing the hair gel from the hair, the hair is manipulated into a desired shape or style and dried.

20. A method for retaining curl definition comprising applying the composition of claim 1 to the hair, curling the hair without rinsing the composition from the hair, and retaining the curl definition of the hair.
